(19) United States
(12) Patent Application Publication
(10) Pub. No.: US 2009/0227758 A1
(43) Pub. Date: Sep. 10, 2009

(54) POLYURETHANES HAVING LOW LEVELS OF ALDEHYDE EMISSIONS

(76) Inventor: Yoshiaki Miyazaki, Yamanashi (JP)

Correspondence Address: The Dow Chemical Company;Gary C. Cohn, P. O. Box 313, Huntingdon Valley, PA, 19006, US

(21) Appl. No.: 12/396,539

(22) Filed: Mar. 3, 2009

Related U.S. Application Data
(60) Provisional application No. 61/034,518, filed on Mar. 7, 2008.

(51) Int. Cl. C07C 209/84; C08G 18/10; C07C 35/00

(52) U.S. Cl. 528/61; 564/498; 568/700

(57) ABSTRACT

Polyols or polyisocyanates, or both, are treated to reduce aldehyde impurities before being reacted together to form a polyurethane. Polyols are treated by adding certain aminoalcohols to them, preferably in the presence of a tertiary amine catalyst. Polyisocyanates are treated by added certain nitroalkanes to them, also preferably in the presence of a tertiary amine catalyst. Polyurethanes made using the treated materials emit smaller quantities of aldehydes.

This application claims priority from U.S. Provisional Patent Application No. 61/034,518, filed Mar. 7, 2008.

This invention relates to polyurethanes which exhibit low levels of aldehyde emissions, and to polyol and isocyanate compositions which are useful to produce such polyurethanes.

Emissions from polymeric materials are a concern in many applications, especially when people or animals are exposed to the polymeric material within an enclosed space. Materials used in workspace, home and automotive environments are a particular concern. Automobile manufacturers are imposing stricter limits on the emissions from polymeric materials that are used in the passenger cabins of cars and trucks. Aldehyde emissions, especially formaldehyde, are a particular cause of concern.

Polyurethanes are used in many office, household and automotive applications. They are used, for example, in appliance applications and as cushioning for bedding and furniture. In automobiles and trucks, polyurethanes are used as seat cushioning, in headrests, in dashboards and instrument panels, in armrests, in headliners, and other applications. These polyurethanes often emit varying levels of formaldehyde.

Formaldehyde scavengers are sometimes used to reduce emissions from various types of materials. JP 2005-154599 describes the addition of an alkali metal borohydride to a polyurethane formulation for that purpose. U.S. Pat. No. 5,506,329 describes the use of nitroalkanes and aminoalcohols as formaldehyde scavengers in textile and plywood applications. That patent also describes certain aldimine oxazolidine compounds as being useful for scavenging formaldehyde from polyisocyanate-containing preparations. U.S. Pat. No. 6,646,034 describes adding various formaldehyde scavengers to a polyacetal resin. These polyacetal resins can engage in various reactions to release formaldehyde. Among the formaldehyde scavengers described there are organic compounds having amino or imino groups, including certain aminoalcohol compounds.

An inexpensive and effective method to reduce aldehyde emissions from polyurethanes is highly desired. Preferably, this method does not result in a significant change in the properties or performance of the polyurethane, and does not produce other fugitive species which can be emitted to the atmosphere or migrate to the surface of the polyurethane part.

In one aspect, this invention is a method comprising mixing an oxazolidine-forming aminoalcohol with a polyol or polyamine containing one or more aldehyde impurities, and subjecting the resulting mixture to conditions such that at least a portion of the aldehyde impurities in the polyol or polyamine react with the aminoalcohol to reduce the level of aldehyde impurities in the polyol or polyamine.

The polyol so produced can be formed into a polyurethane and/or polyurea by reaction with a polyisocyanate, to form a polyurethane and/or polyurea having reduced aldehyde emissions.

In another aspect, the invention is a method comprising mixing a nitroalkane compound with an organic isocyanate containing one or more aldehyde impurities, and subjecting the resulting mixture to conditions such that at least a portion of the aldehyde impurities in the organic isocyanate react with the nitroalkane to reduce the level of aldehyde impurities in the organic isocyanate.

The organic isocyanate so produced can be formed into a polyurethane and/or polyurea by reaction with one or more isocyanate-reactive compounds, to form a polyurethane and/or polyurea having reduced aldehyde emissions.

The invention is also a process for reducing aldehyde emissions from a polyurethane, comprising:
a) mixing an oxazolidine-forming aminoalcohol with a polyol or polyamine containing one or more aldehyde impurities, and subjecting the resulting mixture to conditions such that at least a portion of the aldehyde impurities in the polyol or polyamine react with the aminoalcohol to reduce the level of aldehyde impurities in the polyol or polyamine having a;
b) mixing a nitroalkane compound with a polyisocyanate containing one or more aldehyde impurities, and subjecting the resulting mixture to conditions such that at least a portion of the aldehyde impurities in the polyisocyanate react with the nitroalkane to reduce the level of aldehyde impurities in the polyisocyanate, and then
c) reacting the polyol or polyamine product from step a) with the polyisocyanate product from step b) to form a polyurethane and/or polyurea polymer.

The invention provides an inexpensive and effective way to reduce aldehyde emissions, especially formaldehyde emissions, from a polyurethane and/or polyurea polymer. Either or both of the main precursor materials are treated with a particular type of agent, which reacts with an aldehyde impurity in each case. In the case of a polyol or polyamine starting material, the agent is an aminoalcohol such as is more fully described below. A polyisocyanate is treated with a nitroalkane, again as more fully described below. The respective agents remove aldehydes from the precursor materials, and this reduction of aldehyde levels in the precursors leads to reduced aldehyde emissions when the precursors are formed into polyurethane and/or polyurea polymers. As explained more fully below, in preferred embodiments, the aldehydes are believed to be converted to reactive species that become incorporated into the polymer structure and are rendered non-fugitive.

In certain aspects of the invention, the aldehyde content of a polyol or polyamine is reduced through treatment with an amino alcohol.

The polyol or polyamine can be any material having, on average, at least 1.5 hydroxyl, primary amine and/or secondary amine groups per molecule. The polyol or polyamine preferably has an average of at least 1.8 hydroxyl, primary amine and/or secondary amine groups per molecule. It may have an average of up to 8 or more hydroxyl, primary amine or secondary amine groups per molecule.

Polyols that do not contain primary or secondary amino groups are preferred.

The weight per hydroxyl, primary amino and/or secondary amino group may range from about 30 to 5000 daltons or more. Some polyols and polyamines of interest have a weight per hydroxyl, primary amino and/or secondary amino group of at least 300 daltons and or at least 500 daltons. The weight is may be up to 3000 daltons or up to 2500 daltons.

Suitable polyols include compounds such as alkylene glycols (e.g., ethylene glycol, propylene glycol, 1,4-butane diol, 1,6-hexanediol and the like), glycol ethers (such as diethylene glycol, triethylene glycol, dipropylene glycol, tripropylene glycol and the like), glycerine, trimethylolpropane, tertiary amine-containing polyols such as triethanolamine, triisopropanolamine, ethylene oxide and/or propylene oxide adducts of ethylene diamine, toluene diamine and the like, polyether polyols, polyester polyols, and the like.

Among the suitable polyether polyols are polymers of alkylene oxides such as ethylene oxide, propylene oxide and 1,2-butylene oxide or mixtures of such alkylene oxides. Preferred polyethers are polypropylene oxides or random copolymers of a mixture of propylene oxide and a small amount (up to about 12 weight percent) ethylene oxide. These preferred polyethers can be capped with up to about 30% by weight ethylene oxide.

Polyester polyols are also suitable. These polyester polyols include reaction products of polyols, preferably diols, with polycarboxylic acids or their anhydrides, preferably dicarboxylic acids or dicarboxylic acid anhydrides. The polycarboxylic acids or anhydrides may be aliphatic, cycloaliphatic, aromatic and/or heterocyclic and may be substituted, such as with halogen atoms. The polycarboxylic acids may be unsaturated. Examples of these polycarboxylic acids include succinic acid, adipic acid, terephthalic acid, isophthalic acid, trimellitic anhydride, phthalic anhydride, maleic acid, maleic acid anhydride and fumaric acid. The polyols used in making the polyester polyols preferably have an equivalent weight of about 150 or less and include ethylene glycol, 1,2- and 1,3-propylene glycol, 1,4- and 2,3-butane diol, 1,6-hexane diol, 1,8-octane diol, neopentyl glycol, cyclohexane dimethanol, 2-methyl-1,3-propane diol, glycerine, trimethylol propane, 1,2,6-hexane triol, 1,2,4-butane triol, trimethylolethane, pentaerythritol, quinitol, mannitol, sorbitol, methyl glycoside, diethylene glycol, triethylene glycol, tetraethylene glycol, dipropylene glycol, dibutylene glycol and the like. Polycaprolactone polyols such as those sold by The Dow Chemical Company under the trade name “Tone” are also useful.

Suitable polyamines include aliphatic polyamines such as aminoethylpiperazine, diethylene triamine, triethylene tetraamine and tetraethylenepentaamine, and the so-called aminated polyethers in which all or a portion of the hydroxyl groups of a polyether polyol are converted to primary or secondary amine groups. Suitable such aminated polyethers are sold by Huntsman Chemicals under the trade name JEFFAMINE®. Typical conversions of hydroxyl to amine groups for these commercial materials range from about 70-95%, and thus these commercial products contain some residual hydroxyl groups in addition to the amine groups.

Other suitable polyamines include aromatic polyamines such as toluene diamine, diethyltoluenediamine, methylenediphenyldiamine and aromatic amine-terminated polyethers.

Polyols or polyamines having dispersed polymer particles can also be used. These so-called polymer polyols contain, for example, particles of vinyl polymers such as styrene, acrylonitrile or styrene-acrylonitrile, particles of a polyurea polymer, or polymers of a polyurethane-urea polymer.

Mixtures of two or more polyol and/or polyamine compounds can be treated with the aminoalcohol. In this way, aldehydes can be removed simultaneously from multiple polyol or polyamine materials. In addition, formulated mixtures containing one or more polyols or polyamines, together with, for example, surfactant(s), catalyst(s), blowing agents(s), and/or other additives useful in making a polyurethane can in many cases be treated. In that way, aldehydes can be removed simultaneously from all components of the mixture. The mixture should not include aldehydes (other than as impurities) or other materials that might react with the aminoalcohol in an undesirable way.

Some mixtures of particular interest that can be treated in accordance with an aminoalcohol in accordance with the invention include the following:

A. A mixture including two or more polyether polyols and/or polyester polyols, each having an equivalent weight of 300 to 5000.

B. A mixture including at least one polyether polyol and/or polyester polyol having an equivalent weight of from 300 to 5000, with at least one polyol or polyamine having an equivalent weight of from 30 to 299, especially from 30 to 100.

C. A mixture as in A or B, which further contains at least one catalyst for the reaction of a polyol, polyamine or water with an isocyanate group. The catalyst is preferably an organotin catalyst, and is more preferably a tertiary amine catalyst or mixture of at least one tertiary amine catalyst with at least one organotin catalyst.

D. A mixture as in A, B, or C, which further contains at least one surfactant. The surfactant is preferably an organosilicone surfactant.

E. A mixture as in A, B, C or D, which further contains a blowing agent. Physical blowing agents such as low-boiling hydrocarbons, hydrofluorocarbons, hydrochlorofluorocarbons and the like are suitable. Chemical blowing agents such as carbamate compounds can be used. Water is an especially preferred blowing agent.

In each of mixtures A-E, a preferred amount of the aminoalcohol is from 1 to 50, especially from 1 to 20, and even more preferably from 1 to 10 parts by weight of aminoalcohol per million parts by weight of the mixture.

The starting polyol or polyamine is treated with an oxazolidine-forming aminoalcohol. The aminoalcohol contains at least one primary or secondary amine group and at least one hydroxyl group, and is capable of reacting with an aldehyde group to form an oxazolidine compound. The amine group or groups are preferably primary amine groups. It is preferred that at least one primary or secondary amino group and one hydroxyl group are bonded to adjacent carbon atoms. The amino group may be bonded to a carbon atom that contains one, two or three hydroxymethyl substituents. The amino group is most preferably bonded to a tertiary carbon atom. The aminoalcohol preferably contains at least three carbon atoms, and more preferably contains at least four carbon atoms. Examples of suitable aminoalcohols include 2-amino-2-methyl-1-propanol, 2-amino-2-ethyl-1,3-propandiol and tris(hydroxymethyl) aminomethane.

Preferred oxazolidine-forming aminoalcohols form an oxazolidine compound that contains at least one isocyanate-reactive group, such as a hydroxyl group, a primary amino group or a secondary amino group. Aminoalcohols of this type include materials such as tris(hydroxymethyl) aminomethane.

The polyol or polyamine is treated by mixing the aminoalcohol with it and subjecting the mixture to conditions such that the aminoalcohol reacts with aldehyde species in the polyol or polyamine and thus reduces the concentration of aldehydes in the polyol or polyamine. Often, all that is required is to maintain the mixture for a few hours or a few days at approximately room temperature. A higher temperature can be used if desired to accelerate the removal of aldehydes. Any temperature up to the temperature at which the polyol or polyamine degrades is suitable.

Although the invention is not limited to any theory, it is believed that the aminoalcohol reacts with an aldehyde such as formaldehyde to form an oxazolidine compound. The reaction of formaldehyde with tris(hydroxymethyl)aminomethane is illustrative:

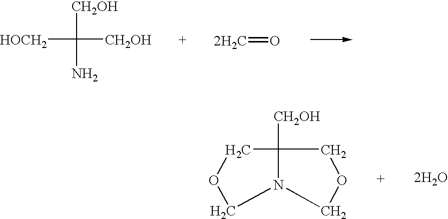

As shown, an amino group and a hydroxyl group can react with formaldehyde to form an oxazolidine ring. Where, as here, the aminoalcohol contains a primary amino group and at least two nearby hydroxyl groups, it can react bifunctionally with two moles of an aldehyde to form a reaction product that contains two fused oxazolidine rings. Note that when the aminoalcohol contains more hydroxyl groups than amine hydrogen atoms, as shown, the reaction product can contain one or more free hydroxyl groups. The free hydroxyl groups allow the reaction product to react further with a polyisocyanate. In this way, the reaction product of the aminoalcohol and the aldehyde can become bound into the structure of a polyurethane polymer. A similar effect can be seen if the aminoalcohol contains more amine hydrogen atoms than hydroxyl groups (in this case forming one or more amino group on the oxazolidine compound), or when the aminoalcohol contains hydroxyl or amino groups which do not engage in an oxazolidine-forming reaction. When bound into the polyurethane in this manner, the reaction product cannot be emitted as a gas from the polyurethane, or migrate to its surface.

The removal of aldehydes from the polyol or polyamine may proceed faster under basic conditions. Therefore, it may be desirable to add a base to the mixture to speed the reaction, if the polyol or polyamine is not itself a basic material. A preferred type of base is a tertiary amine compound, especially a tertiary amine compound that is also a catalyst for the reaction of a polyisocyanate with a polyol or with water. Examples of suitable tertiary amines include, for example, trimethylamine, triethylamine, N-methylmorpholine, N-ethylmorpholine, N,N-dimethylbenzylamine, N,N-dimethylethanolamine, N,N,N′,N′-tetramethyl-1,4-butanediamine, N,N-dimethylpiperazine, 1,4-diazobicyclo-2,2,2-octane, bis(dimethylaminoethyl)ether, bis(2-dimethylaminoethyl)ether, morpholine, 4,4′-(oxydi-2,1-ethanediyl)bis and triethylenediamine. The catalyst may remain in the treated polyol or polyamine until the treated polyol or polyamine is later processed into a polyurethane.

It is generally sufficient to treat the polyol or polyamine with from 1 to 500 parts by weight of the oxazolidine-forming aminoalcohol per million parts by weight of polyol or polyamine. However, quantities above 100 parts per million are usually not required or preferred. A preferred treatment level is from 1 to 50, especially from 1 to 20, and even more preferably from 1 to 10 parts by weight of aminoalcohol per million parts by weight polyol.

In other embodiments of the invention, aldehydes are removed from an organic isocyanate by treatment with a nitroalkane. The polyisocyanate preferably is an organic polyisocyanate having an average of at least one isocyanate groups per molecule. The organic isocyanate preferably contains an average of from about 1.5 to about 6 isocyanate groups per molecule. The equivalent weight per isocyanate group may be from about 55 to about 4000 or more. A preferred organic isocyanate compound has an equivalent weight per isocyanate group of from about 80 to about 2500. An especially preferred organic isocyanate is a polyisocyanate having an equivalent weight of from about 85 to about 500.

Examples of suitable polyisocyanates include, for example, m-phenylene diisocyanate, 2,4- and/or 2,6-toluene diisocyanate (TDI), the various isomers of diphenylmethanediisocyanate (MDI), the so-called polymeric MDI products (which are a mixture of polymethylene polyphenylene polyisocyanates in monomeric MDI), carbodiimide-modified MDI products (such as the so-called “liquid MDI” products which have an isocyanate equivalent weight in the range of 135-170), hexamethylene-1,6-diisocyanate, tetramethylene-1,4-diisocyanate, cyclohexane-1,4-diisocyanate, hexahydrotoluene diisocyanate, hydrogenated MDI (H₁₂ MDI), isophorone diisocyanate, naphthylene-1,5-diisocyanate, methoxyphenyl-2,4-diisocyanate, 4,4′-biphenylene diisocyanate, 3,3′-dimethyoxy-4,4′-biphenyl diisocyanate, 3,3′-dimethyldiphenyl methane-4,4′-diisocyanate, 4,4′,4″-triphenylmethane diisocyanate, hydrogenated polymethylene polyphenylpolyisocyanates, toluene-2,4,6-triisocyanate and 4,4′-dimethyldiphenylmethane-2,2′,5,5′-tetraisocyanate.

Mixtures of two or more organic isocyanate compounds can be treated in accordance with the invention. In addition, mixtures of one or more isocyanate compounds with one or more other materials can be treated, provided that the other materials do not include aldehyde groups or otherwise react undesirably with the nitroalkane or the polyisocyanate. Examples of such other materials include, for example, surfactants, blowing agents, catalysts and the like.

The nitroalkane is a compound having a nitro (NO₂) group bonded directly to a carbon atom of an alkyl group. The carbon atom carrying the nitro group should also be bonded to at least one hydrogen atom. The length of the alkyl group is not important, except that a greater weight of larger molecules may be required. The alkyl group may be unsubstituted, or may be substituted with any substituent which does not interfere with the action of the nitroalkane to reduce aldehyde levels in the polyisocyanate compound. The alkyl group may be linear or branched. Suitable nitroalkanes include nitromethane, nitroethane, 1-nitropropane, 2-nitropropane, 1-nitrobutane, 2-methyl-1-nitropropane, 1-methyl-1-nitropropane and the like.

Suitable conditions for treating the organic isocyanate are similar to those discussed above with respect to treating the polyol or polyamine. The organic isocyanate and nitroalkane are mixed together for a few hours or a few days at approximately room temperature, or a higher temperature up to the temperature at which the organic isocyanate degrades. If desired, a tertiary amine catalyst, such as described before, can be used to accelerate the removal of the aldehydes from the organic isocyanate. Tertiary amines that also catalyze the reaction of an isocyanate group with a polyol, polyamine or water are preferred, as these can remain with the treated material when it is subsequently reacted to form a polyurethane and/or polyurea.

It is believed that the nitroalkane can react with an aldehyde such as formaldehyde to introduce one or more hydroxyalkyl substituents onto the nitroalkane. The reaction of formaldehyde with nitroethane is illustrative:

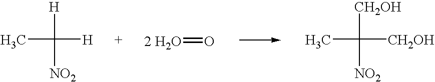

In the foregoing reaction, a single nitroethane molecule is shown to consume two moles of formaldehyde, although in practice the nitroalkane may consume less formaldehyde. The free hydroxyl groups formed in this reaction allow the hydroxyalkylated product to react one or more additional molecules of the organic isocyanate. When the organic isocyanate is a diisocyanate, this reaction can be illustrated as:

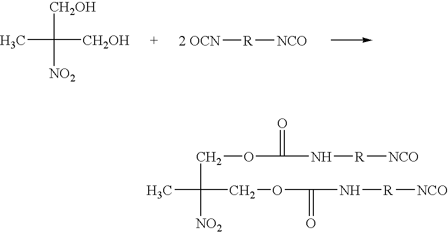

This reaction produces isocyanate-terminated species, when the organic isocyanate contains two or more isocyanate groups. The isocyanate-terminated species can react with a polyol or polyamine to bond it into the structure of a polyurethane or polyurea polymer. As before, this prevents the reaction product from being emitted as a gas from the polyurethane or polyurea polymer and from migrating to the surface.

When the treated polyol or polyamine or the treated polyisocyanate, or both, are used to make a polyurethane and/or polyurea polymer, the polyurethane and/or polyurea polymer exhibits reduced aldehyde emissions, compared to the case when neither the polyol or polyamine nor the polyisocyanate have been treated in accordance with the invention. In preferred embodiments, at least the polyols or polyamines having an equivalent weight of 300 or greater and the polyisocyanate(s) each have been treated in accordance with the invention.

The treated polyisocyanate, polyol or polyamine can be used to make polyurethane and/or polyurea in the same manner as the untreated materials. These methods are well known, and described, for example, in U.S. Pat. Nos. 5,420,170, 5,648,447, 6,107,359, 6,552,100, 6,737,471 and 6,790,872. Various types of polyurethane and/or polyurea polymers can be made, including rigid foams, flexible foams, semi-flexible foams, microcellular elastomers, backings for textiles such as carpeting and other floor coverings, spray elastomers, cast elastomers, polyurethane-isocyanurate foams, reaction injection molded polymers, structural reaction injection molded polymers and the like.

The invention is of particular interest in foamed polyurethanes. Because of the high surface areas of these materials and, in many cases, the ability for gasses to flow in and out of the cells of the foam, foamed polyurethanes sometimes tend to exhibit significant aldehyde emissions, unless measures are taken to abate those emissions.

Particular foam applications of interest include foams for cushioning applications such as bedding and seating and foams for use in automotive interiors, such as flexible and semi-flexible foams for automotive seating, in headrests, in dashboards and instrument panels, in armrests or in headliners.

The polyurethanes are prepared by bringing together one or more polyols and/or polyamines with at least one polyisocyanate, and subjecting the resulting reaction mixture to conditions sufficient to cause the polyisocyanate to react with the polyol(s) and/or polyamine(s) (and water, if present). The components may be heated prior to mixing them to form the reaction mixture. In other cases, the components are mixed at ambient temperatures (such as from 15-40° C.). Heat may be applied to the reaction mixture, but this is often unnecessary. When making a foam, the foam can be made in a free-rise (slabstock) process, in which the foam is free to rise under minimal or no vertical constraint. Alternatively, a molded foam can be made by introducing the reaction mixture in a closed mold and allowing it to foam within the mold. The particular polyol(s), polyamine(s) and polyisocyanate(s) are selected with the desired characteristics of the resulting polyurethane and/or polyurea polymer in mind. Other additives, such as surfactants, catalysts and blowing agents, among others, may be included in the reaction mixture as needed or desired to produce a particular type of foam.

The ratios of the polyisocyanate and polyol components are advantageously selected so as to provide a desired isocyanate index (ratio of NCO to isocyanate-reactive groups). A suitable isocyanate index will depend somewhat on the type of polyurethane and/or polyurea polymer being made. For most applications an isocyanate index of at least 0.7, preferably at least 0.85 and more preferably at least 0.95 is suitable. The isocyanate index may be as high as 5 or more, but more typically it is up to about 1.5, preferably to about 1.35, more preferably to about 1.25.

A catalyst will be used in most cases. Most typically, this catalyst will be incorporated into the polyol component, but in some cases can be mixed into the polyisocyanate component or added as a separate stream. As already mentioned, certain tertiary catalysts that are used in the treatment of the polyisocyanate(s), polyol(s) or polyurea(s) may be carried through into the polyurethane-forming reaction.

Suitable catalysts include those described by U.S. Pat. No. 4,390,645, which is incorporated herein by reference. Representative catalysts include:
(a) tertiary amines, such as trimethylamine, triethylamine, N-methylmorpholine, N-ethylmorpholine, N,N-dimethylbenzylamine, N,N-dimethylethanolamine, N,N,N′, N′-tetramethyl-1,4-butanediamine, N,N-dimethylpiperazine, 1,4-diazobicyclo-2,2,2-octane, bis(dimethylaminoethyl)ether, bis(2-dimethylaminoethyl)ether, morpholine, 4,4′-(oxydi-2,1-ethanediyl)bis and triethylenediamine;
(b) tertiary phosphines, such as trialkylphosphines and dialkylbenzylphosphines;
(c) chelates of various metals, such as those which can be obtained from acetylacetone, benzoylacetone, trifluoroacetyl acetone, ethyl acetoacetate and the like with metals such as Be, Mg, Zn, Cd, Pd, Ti, Zr, Sn, As, Bi, Cr, Mo, Mn, Fe, Co and Ni;
(d) acidic metal salts of strong acids, such as ferric chloride, stannic chloride, stannous chloride, antimony trichloride, bismuth nitrate and bismuth chloride;
(e) strong bases, such as alkali and alkaline earth metal hydroxides, alkoxides and phenoxides;
(f) alcoholates and phenolates of various metals, such as Ti(OR)₄, Sn(OR)₄ and Al(OR)₃, wherein R is alkyl or aryl, and the reaction products of the alcoholates with carboxylic acids, beta-diketones and 2-(N,N-dialkylamino)alcohols;
(g) salts of organic acids with a variety of metals, such as alkali metals, alkaline earth metals, Al, Sn, Pb, Mn, Co, Ni and Cu including, for example, sodium acetate, stannous octoate, stannous oleate, lead octoate, metallic driers, such as manganese and cobalt naphthenate; and
(h) organometallic derivatives of tetravalent tin, trivalent and pentavalent As, Sb and Bi and metal carbonyls of iron and cobalt.

In addition, the reaction mixture can contain various auxiliary components such as surfactants, fillers, colorants, odor masks, flame retardants, biocides, antioxidants, UV stabilizers, antistatic agents, thixotropic agents and cell openers.

Aldehyde emissions from the polyurethane and/or polyurea polymer can be measured by collecting gas samples and analyzing for the aldehyde using any suitable analytic method. Liquid chromatography methods are useful, especially for formaldehyde detection. One standard test method that can be used is Toyota method TSM 0508.

The following examples are provided to illustrate the invention, but are not intended to limit the scope thereof. All parts and percentages are by weight unless otherwise indicated.

### EXAMPLE 1

Five parts per million of nitroethane are added with stirring to an 20/80 mixture of toluene diisocyanate and methylene diphenyl diisocyanate. 500 parts by weight of triethylene diamine are then added per million parts by weight of the diisocyanate. The resulting material is allowed to sit at about 25° C. for 2-3 days.

Five parts per million of tris(hydroxymethyl)aminomethane is added to a formulated polyol component that contains 100 parts by weight of a mixture of polyether polyols, 0.5 parts diethanolamine, 0.4 parts glycerine, 1 part of a cell opener, 3 parts water, 0.1 part of a 33% triethylenediamine solution, 0.1 part of another tertiary amine catalyst and 0.8 part of an organosilicone surfactant. The resulting mixture is allowed to remain at about 25° C. for seven days.

A box foam is prepared by mixing the treated polyisocyanate mixture with the treated formulated polyol component. The proportions are selected to provide an isocyanate index of 1.00. The starting materials are mixed at about 25° C. for 5 seconds using a hand-held mixer, and poured into a mold that is heated to 55° C. The resulting foam is removed from the mold after six minutes and crushed to open the cells. The crushed foam is cut into 100 mm×80 mm×50 mm samples, which are immediately covered with aluminum foil and then placed into a polyethylene bag to retain the volatiles. The samples are kept in this manner for 2 weeks at about 25° C. The foams are then removed from their wrappings, placed in new plastic bags and heated at 60° C. for two hours.

The plastic bags containing the foams are removed from the oven. A measured amount of nitrogen is used to purge the plastic bags. The nitrogen and atmosphere from the plastic bags are captured in a four-liter dinitrophenylhydrazine cartridge. The gas is analyzed for formaldehyde by liquid chromatography, according to Toyota method TSM 0508. Each test piece is found to have released 0.158 micrograms of formaldehyde.

### EXAMPLE 2 AND COMPARATIVE EXAMPLE A

Example 2 is prepared and tested in the same manner as Example 1, except this time only the polyisocyanate mixture is treated. This time, each test piece releases 0.123 micrograms of formaldehyde.

Comparative Sample A is prepared and tested in the same manner as Examples 1 and 2, except this time neither the polyisocyanate mixture nor the formulated polyol is treated. Each test piece is found to release 0.206 micrograms of formaldehyde.

As can be seen by comparing the results from Example 1, Example 2 and Comparative Sample A, formaldehyde emissions are reduced from 23 to 40% through the treatment of the invention.

### EXAMPLES 3 AND 4, AND COMPARATIVE SAMPLE B

Example 3 is prepared and tested in the same manner as Example 1, except that the formulated polyol composition contains 100 parts of a polyol blend, 3 parts of water, 0.25 parts of 33% triethylenediamine solution and 0.7 parts of a mixture of organosilicone surfactants. Each test piece is found to have released 0.125 micrograms of formaldehyde.

Example 4 is a repeat of Example 3, except that only the polyisocyanate composition is treated. Each test piece is found to have released 0.243 micrograms of formaldehyde.

Comparative Sample B is another repetition of Example 3, except this time neither the polyisocyanate mixture nor the formulated polyol composition is treated. In this case, each test piece is found to have released 0.321 micrograms of formaldehyde.

In these three runs, formaldehyde emissions are reduced from 24 to 61% by treatment according to the invention.

What is claimed is:

1. A method comprising mixing an oxazolidine-forming aminoalcohol with a polyol or polyamine containing one or more aldehyde impurities, and subjecting the resulting mixture to conditions such that at least a portion of the aldehyde impurities in the polyol or polyamine react with the aminoalcohol to reduce the level of aldehyde impurities in the polyol or polyamine.

2. The method of claim 1, wherein the oxazolidine-forming aminoalcohol reacts with an aldehyde compound to form an oxazolidine compound having at least one hydroxyl, primary amino or secondary amino group.

3. The method of claim 2 wherein the oxazolidine-forming aminoalcohol contains at least one primary or secondary amino group and at least one hydroxyl group bonded to adjacent carbon atoms.

4. The method of claim 3 wherein the oxazolidine-forming aminoalcohol contains at least one primary or secondary amino group bonded to a carbon atom that contains one, two or three hydroxymethyl substituents.

5. The method of claim 3 wherein the primary or secondary amino group is bonded to a tertiary carbon atom.

6. The method of claim 2, wherein the mixture of the aminoalcohol and the polyol and/or polyamine further contains at least one tertiary amine catalyst for a reaction between a polyisocyanate and water, a polyol or a polyamine.

7. The method of any claim 1, further comprising mixing the polyol or polyamine having a reduced level of aldehyde impurities with a polyisocyanate under conditions sufficient to produce a polyurethane and/or polyurea having reduced aldehyde emissions.

8. A method comprising mixing a nitroalkane compound with an organic isocyanate containing one or more aldehyde impurities, and subjecting the resulting mixture to conditions such that at least a portion of the aldehyde impurities in the organic isocyanate compound react with the nitroalkane to reduce the level of aldehyde impurities in the organic isocyanate compound.

9. The method of claim 8 wherein the nitroalkane includes at least one nitro group bonded to a carbon atom of an alkyl group, and the carbon atom is bonded to at least one hydrogen atom.

10. The method of claim 9, wherein the nitroalkane is one or more of nitromethane, nitroethane, 1-nitropropane and 2-nitropropane, 1-nitrobutane, 2-methyl-1-nitropropane or 1-methyl-1-nitropropane.

11. The method of claim 8, wherein the mixture of the nitroalkane and the organic isocyanate compound further contains at least one tertiary amine catalyst for a reaction between a polyisocyanate and water, a polyol or a polyamine.

12. The method of claim 8, further comprising mixing the polyisocyanate having a reduced level of aldehyde impurities with a polyol and/or polyamine under condition sufficient to produce a polyurethane and/or polyurea having reduced aldehyde emissions.

13. A process for reducing aldehyde emissions from a polyurethane, comprising:
a) mixing an oxazolidine-forming aminoalcohol with a polyol or polyamine containing one or more aldehyde impurities, and subjecting the resulting mixture to conditions such that at least a portion of the aldehyde impurities in the polyol or polyamine react with the aminoalcohol to reduce the level of aldehyde impurities in the polyol or polyamine;
b) mixing a nitroalkane compound with a polyisocyanate containing one or more aldehyde impurities, and subjecting the resulting mixture to conditions such that at least a portion of the aldehyde impurities in the polyisocyanate react with the nitroalkane to reduce the level of aldehyde impurities in the polyisocyanate, and then
c) reacting the polyol or polyamine product from step a) with the polyisocyanate product from step b) to form a polyurethane and/or polyurea polymer.

14. The method of claim 13 wherein the oxazolidine-forming aminoalcohol contains at least one primary or secondary amino group and at least one hydroxyl group bonded to adjacent carbon atoms.

15. The method of claim 14 wherein the oxazolidine-forming aminoalcohol contains at least one primary or secondary amino group bonded to a carbon atom that contains one, two or three hydroxymethyl substituents.

16. The method of claim 14 wherein the primary or secondary amino group is bonded to a tertiary carbon atom.

17. The method of claim 13, wherein the oxazolidine-forming aminoalcohol reacts with an aldehyde compound to form an oxazolidine compound having at least one hydroxyl, primary amino or secondary amino group.

18. The method of claim 17 wherein the nitroalkane includes at least one nitro group bonded to a carbon atom of an alkyl group, and the carbon atom is bonded to at least one hydrogen atom.

19. The method of claim 18, wherein the nitroalkane is one or more of nitromethane, nitroethane, 1-nitropropane and 2-nitropropane, 1-nitrobutane, 2-methyl-1-nitropropane or 1-methyl-1-nitropropane.
